(19) United States
(12) Patent Application Publication
(10) Pub. No.: US 2013/0203811 A1
(43) Pub. Date: Aug. 8, 2013

(54) THALIDOMIDE AND THALIDOMIDE ANALOGUES FOR THE PREVENTION AND TREATMENT OF SARCOPENIA

(71) Applicant: Patrick T. PRENDERGAST, Byrock (AU)

(72) Inventor: Patrick T. PRENDERGAST, Byrock (AU)

(21) Appl. No.: 13/446,608

(22) Filed: Apr. 13, 2012

(30) Foreign Application Priority Data
Feb. 8, 2012 (IE) 2012/0053

(51) Int. Cl. A61K 31/454; A61P 21/00

(52) U.S. Cl. 514/323

(57) ABSTRACT

The present invention relates to thalidomide and/or thalidomide analogues and methods for using same to maintain or increase muscle mass to prevent and/or treat sarcopenia. The invention also provides pharmaceutical compositions comprising thalidomide and thalidomide analogues for the prevention and/or treatment of sarcopenia.

### CROSS-REFERENCE TO RELATED APPLICATION

This application claims the benefit of Irish Provisional Patent Application No. 2012/0053, filed Feb. 8, 2012, the entirety of which is incorporated herein by reference as if set forth in its entirety.

### DESCRIPTION

The present invention relates to thalidomide and thalidomide analogues and methods for using same to maintain or increase muscle mass. In particular the present invention provides compositions and methods to prevent and/or treat sarcopenia.

Muscle atrophy (also known as muscle wasting) is a debilitating syndrome which slowly develops with age (sarcopenia) or rapidly appears at the late stages of deadly diseases such as cancer, AIDS and sepsis (cachexia). Despite the prevalence and the drastic detrimental effects of these two conditions, there are currently no widely used, effective treatment options for those suffering from muscle wasting. Rosenberg first coined the term Sarcopenia, from the Greek, which literally means poverty of flesh, to describe age-associated loss of skeletal muscle mass. Sarcopenia is now generally used to describe age-related changes that occur within skeletal muscle and thus encompasses the effects of altered central and peripheral nervous system innervations, altered hormone status, inflammatory effects and altered caloric and protein intake.

Sarcopenia is characterised by the loss of skeletal muscle mass as well as strength, these morphological and functional modifications result from intrinsic events, such as changes in the muscle fibre type composition, mitochondrial dysfunction and oxidative damage, and from extrinsic factors including reduced physical activities and excessive and/or unbalanced nutritional intake. Sarcopenia has important health consequences for older adults because it is associated with an increased risk of falls, hip fractures, bone mineral loss and physical disability.

Baumgartner et al. (Am J Epidemiol 1998; 147:755-63; 149:111) have defined sarcopenia as appendicular skeletal muscle mass (kg/height2 (m2)) being less than two standard deviations below the mean of a young reference group. This is referred to as a ‘t-score’ hereinafter. Baumgartner et al used the data from the New Mexico Elder Health Survey 1993-1995, to develop a method for estimating the prevalence of sarcopenia and found that the prevalence increased from 13-24% in persons under 70 years of age to >50% in persons over 80 years of age. The study by Baumgartner et al was one of the first to estimate the extent of the prevalence of sarcopenia.

A t-score is determined by measuring the axial skeletal muscle mass of a patient, typically by dxa (i.e. dual energy xray absorptiometry) or a similar and reducible measure. The measurement of axial skeletal muscle mass can be used to follow the progress of the patient to determine if treatment is slowing, preventing or reversing muscle mass decline.

Multiple factors appear to be involved in the development of sarcopenia however no primary cause has been identified as yet. Although the overall biological mechanism of sarcopenia is not fully understood, observational studies have shown that satellite cells which are involved in muscle regeneration are much lower in older people and therefore, could play a role in sarcopenia. Other factors including hormonal changes, such as growth hormone (GH) and insulin-like growth factor (IGF-1) and androgens which help regulate growth and development of skeletal muscle appear to decrease in old age. It has also been suggested that the renin-angiotensin system may play a role in modulating muscle function.

Sarcopenia is a major public health problem in industrialised nations, placing an increasing burden on public healthcare systems because the loss of skeletal muscle mass and strength that characterises this indication increases the dependence and the risk of injury caused by sudden falls in elderly people.

It has been estimated that up to 15% of people older than 65 years and as many as 50% of people older than 80 years have sarcopenia. Sarcopenia has a major impact on public health and the cost in the United States alone was estimated to be $18.5 billion in 2000. With the rising number of older people worldwide, the cost is ever increasing.

Treating sarcopenia includes slowing its progression, stopping its progression and partially reversing it.

An example of slowing the progression of sarcopenia would be to change the length of time a patient would go from a t-score of −1.5 to −2 (e.g. if such a progress ion would normally take 5 years, then treating as used herein could slow this change to 10 years). Examples of partial reversal include reducing a t-score 0.1, 0.2, 0.3, 0.4, 0.5, 0.6, 0.7, 0.8, 0.9, 1.0 or more units (e.g. moving from a t-score of −2 to a t-score of −1.9, −1.8, −1.7, −1.6, −1.5, −1.4, −1.3, −1.2, −1.1 etc). Treating sarcopenia also includes delaying the onset of sarcopenia. For example, if a typical male age 50 would begin to see signs of sarcopenia by age 55, treatment according to the present invention could delay the onset 1, 2, 3, 4, 5, 6, 7, 8, 9, 10 or more years. Thus, treating sarcopenia would include treating patients who have not yet been diagnosed with sarcopenia, but who would be vulnerable or expected to be vulnerable to developing sarcopenia. Patients who are vulnerable also include (a) patients using glucocorticoid steroids, (b) patients with chronic infections, (c) patients with chronic inflammatory conditions (e.g. inflammatory bowel disease), and (d) patients with cancer.

Another type of patient that would benefit from the present invention is one that has suffered some loss of muscle mass, but who does not suffer from a condition that interferes with acts of daily living and/or prevents the subject from living an independent life (e.g. a patient who might soon need assisted living).

In one embodiment, a further decline in t-score is prevented via treatment for at least one year.

In another embodiment, an increase in the t-score of a patient is obtained via treatment for at least one year.

Examples of t-scores include 3, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0, −0.1, −0.2, −0.3, −0.4, −0.5, −0.6, −0.7, −0.8, −0.9, −1.0, −1.1, −1.2, −1.3, −1.4, −1.5, −1.6, −1.7, −1.8, −1.9, −2.0, −2.1, −2.2, −2.3, −2.4, −2.5, −2.6, −2.7, −2.8, −2.9, −3.0, −3.1, −3.2, −3.3, −3.4, −3.5, −3.6, −3.7, −3.8, −3.9, −4.0, −4.1, −4.2, −4.3, −4.4, −4.5, −4.6, −4.7, −4.8, −4.9, −5.0, −5.1, −5.2, −5.3, −5.4, −5.5, −5.6, −5.7, −5.8, −5.9, and −6.0. Typically patients with negative t-scores are more likely to be treated for sarcopenia. However a patient that is at risk of losing function or who has a medical need to maintain muscle may also be a subject for treatment in accordance with the present invention even if their t-score is 0 or greater.

In another embodiment, the patient has a t-score selected from (a) ≦−3, (b) ≦−2.5, (c) ≦−2, (d) ≦−1.5, (e) ≦−1.0 and (f) ≦−0.5.

The age or age range of the patient can vary depending on their susceptibility to sarcopenia. Examples of ages and age ranges include (a) 40-45, (b) 45-50, (c) 50-55, (d) 55-60, (e) 60-65, (f) 65-70, (g) 70-75, (h) 75-80, (i) 80-85, (j) 85-90 or older.

In another embodiment, the age of the patient is selected from at least (a) 40, (b) 50, (c) 60, (e) 65 and (f) 70.

In another embodiment, the present invention provides a novel method of maintaining and/or increasing muscle mass to treat sarcopenia, comprising: administering to a patient in need thereof a therapeutically effective amount of a pharmaceutical composition, comprising thalidomide, or an analogue of thalidomide and a pharmaceutically acceptable carrier.

Thalidomide (N-α-phthalimidoglutarimide) is a glutamic acid derivative that was introduced onto the market as a sedative hypnotic in 1956, but was withdrawn in 1961 due to the development of severe congenital abnormalities in babies born to mothers using it for morning sickness. Interest in the agent was reawakened after thalidomide was found clinically effective in the treatment of erythema nodosum leprosum (ENL) and in the treatment of HIV wasting syndrome and various cancers. Mechanistic studies of its ENL activity also demonstrated an anti-tumor necrosis factor alpha (anti-TNF-α) action. Specifically, thalidomide enhances the degradation of TNF-α RNA, and thereby lowers its synthesis and secretion. Further studies have defined it to be a co-stimulator of CD8+ and CD4+ T cells, an inhibitor of angiogenesis via its inhibitory actions on basic fibroblast growth factor (bFGF) and vascular endothelial growth factor (VEGF), and an inhibitor of the transcription factor, NFKB. Overproduction of TNF-α has been implicated in many inflammatory diseases, such as rheumatoid arthritis, graft-versus-host disease and Crohn's disease, and it additionally exacerbates ENL, septic shock, AIDS and dementia associated with Alzheimer's disease (AD).

The present invention discloses thalidomide and thalidomide analogues for maintaining and increasing muscle mass in a subject. The invention also discloses compositions and methods for the prevention and/or treatment of sarcopenia. In some embodiments, the disclosed thalidomide analogues are sulfur-analogues of thalidomide, its open-ring metabolites and its derivatives (such as its hydroxylated derivatives) in which one or more carbonyl groups are replaced by thiocarbonyl groups. For example, in some embodiments, thalidomide analogues wherein at least one carbonyl group on the phthaloyl moiety or on the glutaramide moiety (or its open ring form) of a thalidomide or a thalidomide analogue is replaced by a thiocarbonyl group.

Still further, a method for preventing and/or treating sarcopenia in a subject is disclosed. The method includes administering to the subject a therapeutically effective amount of one or more of any of the disclosed compounds. Examples of compounds useful for the method are shown below.

Additionally, the disclosed compounds can be combined with pharmaceutically acceptable excipients, and optionally sustained-release matrices, such as biodegradable polymers, to form therapeutic compositions. Therefore, also disclosed are pharmaceutical compositions including one or more of any of the compounds disclosed below and a pharmaceutically acceptable carrier. The composition may comprise a unit dosage form of the composition, and may further comprise instructions for administering the composition to a subject to prevent and/or treat sarcopenia.

The disclosed pharmaceutical compositions can be in the form of tablets, capsules, powders, granules, lozenges, liquid or gel preparations, such as oral, topical, or sterile parenteral solutions or suspensions (e.g. eye or ear drops, throat or nasal sprays etc), transdermal patches and other forms known in the art.

Pharmaceutical compositions can be administered systemically or locally in any manner appropriate to the treatment of a given condition, including orally, parenterally, rectally, nasally, buccally, vaginally, topically, optically, by inhalation spray, or via an implanted reservoir.

The term ‘parenterally’ as used herein includes, but is not limited to subcutaneous, intravenous, intramuscular, intrasternal, intrasynovial, intrathecal, intrahepatic, intralesional and intracranial administration, for example, by injection of infusion. Pharmaceutically acceptable carriers include, but are not limited to ion exchangers, alumina, aluminum stearate, lecithin, serum proteins (such as human serum albumin), buffers (such as phosphates), glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Disclosed are thalidomide analogues that can be used to maintain and increase muscle mass in order to prevent and/or treat sarcopenia. Pharmaceutically acceptable salts, stereoisomers, and metabolites of all of the disclosed compounds also are contemplated. In some embodiments, the thalidomide analogues are thiothalidomide derivatives in which carbonyl groups in corresponding nonsulfur-containing thalidomide derivatives are replaced by one or more thiocarbonyl groups.

In the structures that follow, all valency requirements are understood to be satisfied. Thus, for example, carbon atoms have four bonds to other atoms, even if all such bonds are not shown. As is understood by those of ordinary skill in the art, where all four bonds to a carbon atom are not shown, additional bonds to hydrogen atoms are implied. Further substitution of such implied hydrogen atoms is possible.

In other embodiments, the disclosed compounds include compounds having the chemical formula:

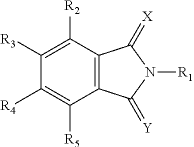

wherein X and Y are independently CH₂, oxygen or sulfur, and at least one of X and Y is sulfur if R₁ does not include a sulfur atom; each of R₂-R₅ are independently hydrogen, hydroxyl, acyl, substituted acyl, acyloxy, substituted acyloxy, alkyl, substituted alkyl, alkenyl, substituted alkenyl, alkynyl, substituted alkynyl, alkoxy, substituted alkoxy, aryl, substituted aryl, amino, substituted amino, halogen, nitro or linked to form a five- or six-membered, unsubstituted or substituted, aliphatic, aromatic or heterocyclic ring, for example, hydrogen, lower alkyl, acyloxy, halogen, hydroxyl, amino or nitro such as hydrogen, acyloxy or hydroxyl; and R₁ is an unsubstituted or substituted, aliphatic or aromatic heterocyclic ring, an unsubstituted or substituted cycloalkenyl ring, or

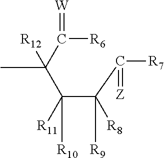

wherein W and Z are each independently oxygen or sulfur, R₆ and R₇ are each independently hydroxyl, alkoxy or substituted alkoxy, and each of R₈-R₁₂ are independently hydrogen, hydroxyl, acyl, substituted acyl, acyloxy, substituted acyloxy, alkyl, substituted alkyl, alkenyl, substituted alkenyl, alkynyl, substituted alkynyl, alkoxy, substituted ally, aryl, substituted aryl, amino, substituted amino, halogen or nitro, for example, hydrogen, lower alkyl, acyloxy, halogen, hydroxyl, amino or nitro such as hydrogen, acyloxy or hydroxyl.

In particular embodiments, R₁ is

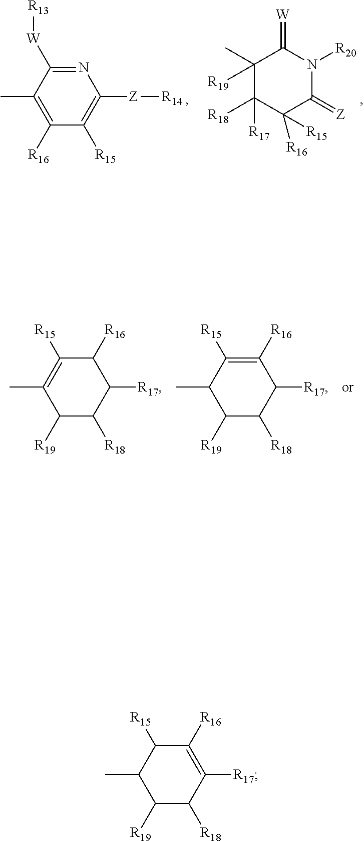

wherein W and Z are each independently oxygen or sulfur, R₁₃ and R₁₄ are each independently hydrogen, alkyl or substituted alkyl; R₂₀ is hydrogen, hydroxyl, alkyl or substituted alkyl such as aryl substituted alkyl; and R₁₅-R₁₉ are each independently hydrogen, hydroxyl, acyl, substituted acyl, acyloxy, substituted acyloxy, alkyl, substituted alkyl, alkenyl, substituted alkenyl, alkynyl, substituted alkynyl, alkoxy, substituted alkoxy, aryl, substituted aryl, amino, substituted amino, halogen or nitro, for example, hydrogen, lower alkyl, acyloxy, halogen, hydroxyl, amino or nitro such as hydrogen, acyloxy or hydroxyl. In some embodiments, at least one of R₂, R₃, R₄, R₅, R₈, R₉, R₁₀, R₁₁, R₁₅, R₁₆, R₁₇, R₁₈ and R₁₉ is hydroxyl. In other embodiments, at least one of X, Y, W and Z is sulfur, at least two of X, Y, W and Z are sulfur, or at least three of X, Y, W and Z are sulfur. For example, in more particular embodiments, X or Y is sulfur, and both W and Z are oxygen if present; both X and Y are sulfur and both W and Z are oxygen if present; X and Y are both oxygen and W or Z is sulfur if present; both X and Y are sulfur and W or Z is sulfur if present; or X or Y are sulfur and both W and Z are sulfur if present. Alternatively, where W and Z are present the following are possible: X═S, Y═O, W═O, Z═O; X═O, Y═S, W═O, Z═O; X═O, Y═O, W═S, Z═O; X═O, Y═O, W═O, Z═S; X═S, Y═S, W═O, Z═O; X═S, Y═O, W═S, Z═O; X═S, Y═O, W═O, Z═S; X═O, Y═O, W═S, Z═S; X═O, Y═S, W═O, Z═S; X═O, Y═S, W═S, Z═O; X═S, Y═S, W═S, Z═O; X═S, Y═S, W═O, Z═S; X═S, Y═O, W═S, Z═S; X═O, Y═S, W═S, Z═S; or X═S, Y═S, W═S, Z═S. In other particular embodiments X═S and Y═CH₂.

In more particular embodiments, the disclosed compounds have the formula

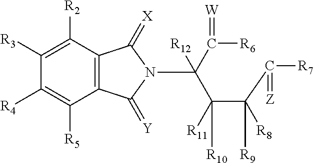

wherein X, Y, W and Z are independently sulfur or oxygen and at least one of X, Y, W and Z is sulfur, and R₂-R₁₂ are as before. For example, in more particular embodiments, X or Y is sulfur, and both W and Z are oxygen if present; both X and Y are sulfur and both W and Z are oxygen if present; X and Y are both oxygen and W or Z is sulfur if present; both X and Y are sulfur and W or Z is sulfur if present; or X or Y are sulfur and both W and Z are sulfur if present. Alternatively, where W and Z are present the following are possible: X═S, Y═O, W═O, Z═O; X═O, Y═S, W═O, Z═O; X═O, Y═O, W═S, Z═O; X═O, Y═O, W═O, Z═S; X═S, Y═S, W═O, Z═O; X═S, Y═O, W═S, Z═O; X═S, Y═O, W═O, Z═S; X═O, Y═O, W═S, Z═S; X═O, Y═S, W═O, Z═S; X═O, Y═S, W═S, Z═O; X═S, Y═S, W═S, Z═O; X═S, Y═S, W═O, Z═S; X═S, Y═O, W═S, Z═S; X═O, Y═S, W═S, Z═S; or X═S, Y═S, W═S, Z═S.

In more particular embodiments, at least one of R₂-R₅ and R₈-R₁₁ is hydroxyl. Specific examples of such compounds include:

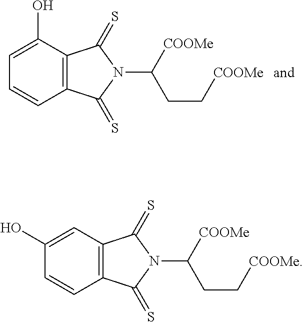 [SMILES: COC(=O)CCC(C(=O)OC)N1C(=S)c2cccc(O)c2C1=S ; COC(=O)CCC(C(=O)OC)N1C(=S)c2ccc(O)cc2C1=S]

In other more particular embodiments, the disclosed compounds have the chemical formula:

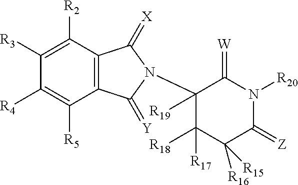

wherein W, X, Y and Z each are independently sulfur or oxygen and at least one of W, X, Y and Z is sulfur; and R₂-R₅, and R₁₅-R₂₀ are as before. For example, in more particular embodiments, X or Y is sulfur, and both W and Z are oxygen; both X and Y are sulfur and both W and Z are oxygen; X and Y are both oxygen and W or Z is sulfur; both X and Y are sulfur and W or Z is sulfur; or X or Y are sulfur and both W and Z are sulfur. Alternatively, the following are possible: X═S, Y═O, W═O, Z═O; X═O, Y═S, W═O, Z═O; X═O, Y═O, W═S, Z═O; X═O, Y═O, W═O, Z═S; X═S, Y═S, W═O, Z═O; X═S, Y═O, W═S, Z═O; X═S, Y═O, W═O, Z═S; X═O, Y═O, W═S, Z═S; X═O, Y═S, W═O, Z═S; X═O, Y═S, W═S, Z═O; X═S, Y═S, W═S, Z═O; X═S, Y═S, W═O, Z═S; X═S, Y═O, W═S, Z═S; X═O, Y═S, W═S, Z═S; or X═S, Y═S, W═S, Z═S. In more particular embodiments, at least one of R₂-R₅, and R₁₅-R₁₉ is hydroxyl. Specific examples of such compounds include:

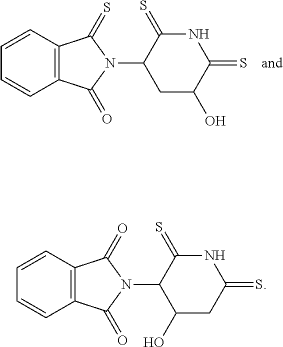 [SMILES: O=C1c2ccccc2C(=S)N1C1CC(O)C(=S)NC1=S ; O=C1c2ccccc2C(=O)N1C1C(=S)NC(=S)CC1O]

The disclosed compounds also include compounds having the formula:

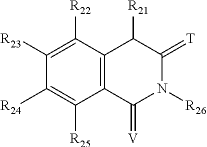

wherein T and V are independently oxygen or sulfur, R₂₁-R₂₅ are independently hydrogen, hydroxyl, acyl, substituted acyl, acyloxy, substituted acyloxy, alkyl, substituted alkyl, alkenyl, substituted alkenyl, alkynyl, substituted alkynyl, alkoxy, substituted alkoxy, aryl, substituted aryl, amino, substituted amino, halogen or nitro, for example, hydrogen, lower alkyl, acyloxy, halogen, hydroxyl, amino or nitro such as hydrogen, acyloxy or hydroxyl; and R₂₆ is

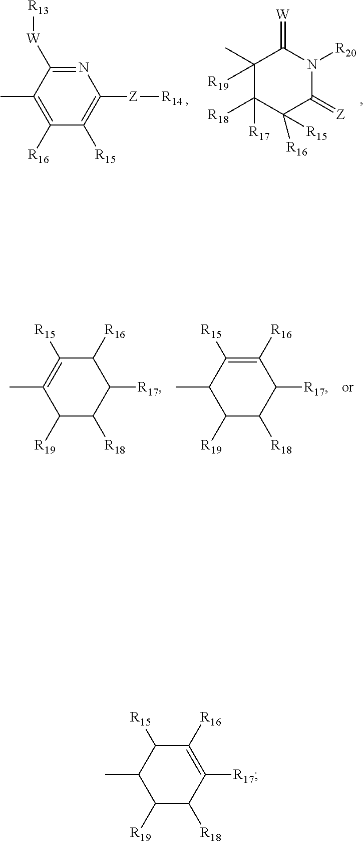

wherein W, Z and R₁₃-R₂₀ are as before. For example, in more particular embodiments, T or V is sulfur, and both W and Z are oxygen if present; both T and V are sulfur and both W and Z are oxygen if present; T and V are both oxygen and W or Z is sulfur if present; both T and V are sulfur and W or Z is sulfur if present; or T or V are sulfur and both W and Z are sulfur if present. Alternatively, where W and Z are present the following are possible: T=O, V═O, W═O, Z═O; T=S, V═O, W═O, Z═O; T=O, V═S, W═O, Z═O; T=O, V═O, W═S, Z═O; T=O, V═O, W═O, Z═S; T=S, V═S, W═O, Z═O; T=S, V═O, W═S, Z═O; T=S, V═O, W═O, Z═S; T=O, V═O, W═S, Z═S; T=O, V═S, W═O, Z═S; T=O, V═S, W═S, Z═O; T=S, V═S, W═S, Z═O; T=S, V═S, W═O, Z═S; T=S, V═O, W═S, Z═S; T=O, V═S, W═S, Z═S; or T=S, V═S, W═S, Z═S. In some embodiments, at least one of R₁₅-R₁₉ and R₂₂-R₂₆ is hydroxyl.

Still further, the disclosed compounds include compounds having the formula:

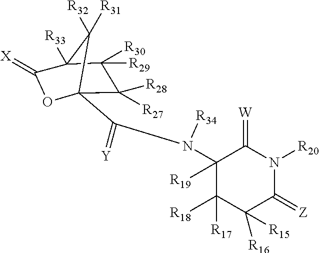

wherein X, Y are each independently oxygen or sulfur, W, X and R₁₅-R₂₀ are as before; R₂₇-R₃₃ are each independently hydrogen, hydroxyl, acyl, substituted acyl, acyloxy, substituted acyloxy, alkyl, substituted alkyl, alkenyl, substituted alkenyl, alkynyl, substituted alkynyl, alkoxy, substituted alkoxy, aryl, substituted aryl, amino, substituted amino, halogen or nitro, for example, hydrogen, lower alkyl, acyloxy, halogen, hydroxyl, amino or nitro such as hydrogen, acyloxy or hydroxyl; and R₃₄ is hydrogen, alkyl or substituted alkyl. For example, in more particular embodiments, X or Y is sulfur, and both W and Z are oxygen; both X and Y are sulfur and both W and Z are oxygen; X and Y are both oxygen and W or Z is sulfur; both X and Y are sulfur and W or Z is sulfur; or X or Y are sulfur and both W and Z are sulfur. Alternatively, the following are possible: X═O, Y═O, W═O, Z═O; X═S, Y═O, W═O, Z═O; X═O, Y═S, W═O, Z═; X═O, Y═O, W═S, Z═O; X═O, Y═O, W═O, Z═S; X═S, Y═S, W═O, Z═O; X═S, Y═O, W═S, Z═O; X═S, Y═O, W═O, Z═S; X═O, Y═O, W═S, Z═S; X═O, Y═S, W═O, Z═S; X═O, Y═S, W═S, Z═O; X═S, Y═S, W═S, Z═O; X═S, Y═S, W═O, Z═S; X═S, Y═O, W═S, Z═S; X═O, Y═S, W═S, Z═S; or X═S, Y═S, W═S, Z═S.

In addition, the disclosed compounds include compounds having the formula:

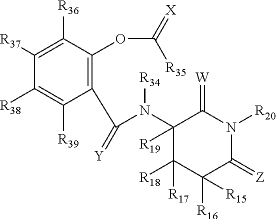

wherein X and Y are each independently oxygen or sulfur; W, Z, R₁₅-R₂₀ and R₃₄ are as before, R₃₅ is alkyl or substituted alkyl, and R₃₆-R₃₉ are each independently hydrogen, hydroxyl, acyl, substituted acyl, acyloxy, substituted acyloxy, alkyl, substituted alkyl, alkenyl, substituted alkenyl, alkynyl, substituted alkynyl, alkoxy, substituted alkoxy, aryl, substituted aryl, amino, substituted amino, halogen or nitro, for example, hydrogen, lower alkyl, acy-loxy, halogen, hydroxyl, amino or nitro such as hydrogen, acyloxy or hydroxyl. For example, in more particular embodiments, X or Y is sulfur, and both W and Z are oxygen; both X and Y are sulfur and both W and Z are oxygen; X and Y are both oxygen and W or Z is sulfur; both X and Y are sulfur and W or Z is sulfur; or X or Y are sulfur and both W and Z are sulfur. Alternatively, the following are possible: X═O, Y═O, W═O, Z═O; X═S, Y═O, W═O, Z═O; X═O, Y═S, W═O, Z═O; X═O, Y═O, W═S, Z═O; X═O, Y═O, W═O, Z═S; X═S, Y═S, W═O, Z═O; X═S, Y═O, W═S, Z═O; X═S, Y═O, W═O, Z═S; X═O, Y═O, W═S, Z═S; X═O, Y═S, W═O, Z═S; X═O, Y═S, W═S, Z═O; X═S, Y═S, W═S, Z═O; X═S, Y═S, W═O, Z═S; X═S, Y═O, W═S, Z═S; X═O, Y═S, W═S, Z═S; or X═S, Y═S, W═S, Z═S.

Other embodiments include compounds having the formula:

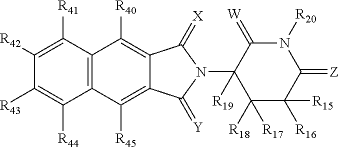

wherein X and Y each are independently oxygen or sulfur; W, Z and R₁₅-R₂₀ are as before; and R₄₀-R₄₅ are each independently hydrogen, hydroxyl, acyl, substituted acyl, acyloxy, substituted acyloxy, alkyl, substituted alkyl, alkenyl, substituted alkenyl, alkynyl, substituted alkynyl, alkoxy, substituted alkoxy, aryl, substituted aryl, amino, substituted amino, halogen or nitro, for example, hydrogen, lower alkyl, acyloxy, halogen, hydroxyl, amino or nitro such as hydrogen, acyloxy or hydroxyl. For example, in more particular embodiments, X or Y is sulfur, and both W and Z are oxygen; both X and Y are sulfur and both W and Z are oxygen; X and Y are both oxygen and W or Z is sulfur; both X and Y are sulfur and W or Z is sulfur; or X or Y are sulfur and both W and Z are sulfur. Alternatively, the following are possible: X═O, Y═O, W═O, Z═O; X═S, Y═O, W═O, Z═O; X═O, Y═S, W═O, Z═O; X═O, Y═O, W═S, Z═O; X═O, Y═O, W═O, Z═S; X═S, Y═S, W═O, Z═O; X═S, Y═O, W═S, Z═O; X═S, Y═O, W═O, Z═S; X═O, Y═O, W═S, Z═S; X═O, Y═S, W═O, Z═S; X═O, Y═S, W═S, Z═O; X═S, Y═S, W═S, Z═O; X═S, Y═S, W═O, Z═S; X═S, Y═O, W═S, Z═S; X═O, Y═S, W═S, Z═S; or X═S, Y═S, W═S, Z═S.

The disclosed compounds further include compounds having the formula:

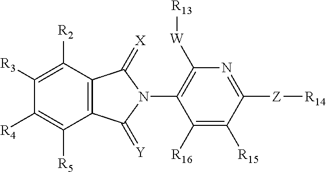

wherein X Y, W and Z are independently oxygen or sulfur, and R₂-R₅ and R₁₃-R₁₆ are as before. For example, in more particular embodiments, X or Y is sulfur, and both W and Z are oxygen; both X and Y are sulfur and both W and Z are oxygen; X and Y are both oxygen and W or Z is sulfur; both X and Y are sulfur and W or Z is sulfur; or X or Y are sulfa and both W and Z are sulfur. Alternatively, the following are possible: X═O, Y═O, W═O, Z═O; X═S, Y═O, W═O, Z═O; X═O, Y═S, W═O, Z═O; X═O, Y═O, W═S, Z═O; X═O, Y═O, W═O, Z═S; X═S, Y═S, W═O, Z═O; X═S, Y═O, W═S, Z═O; X═S, Y═O, W═O, Z═S; X═O, Y═O, W═S, Z═S; X═O, Y═S, W═O, Z═S; X═O, Y═S, W═S, Z═O; X═S, Y═S, W═S, Z═O; X═S, Y═S, W═O, Z═S; X═S, Y═O, W═S, Z═S; X═O, Y═S, W═S, Z═S; or X═S, Y═S, W═S, Z═S.

Also disclosed is a thalidomide analogue compound having the formula:

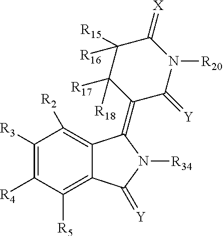

wherein X, Y and Z are independently oxygen or sulfur, and R₂-R₅, R₁₅-R₂₀ and R₃₄ are as before. For example, in more particular embodiments, X or Y is sulfur, and Z is oxygen; both X and Y are sulfur and Z is oxygen; X and Y are both oxygen and Z is sulfur. Alternatively, the following are possible: X═O, Y═O, Z═O; X═S, Y═O, Z═O; X═O, Y═S, Z═O; X═O, Y═O, Z═S; X═S, Y═S, Z═O; X═S, Y═O, Z═S; X═O, Y═S, Z═S; or X═S, Y═S, Z═S.

Also disclosed is a thalidomide analogue compound having the formula:

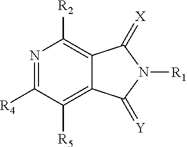

wherein X and Y are independently oxygen or sulfur, and R₁, R₂, R₄ and R₅ are as before. For example, in particular embodiments, R₁ is

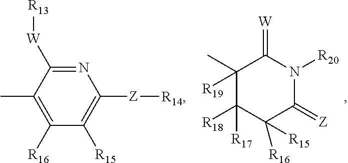

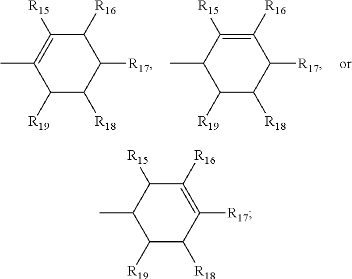

wherein W, Z, and R₁₃-R₂₀ are as before. For example, in more particular embodiments, X or Y is sulfur, and both W and Z are oxygen if present; both X and Y are sulfur and both W and Z are oxygen if present; X and Y are both oxygen and W or Z is sulfur if present; both X and Y are sulfur and W or Z is sulfur if present; or X or Y are sulfur and both W and Z are sulfur if present. Alternatively, where W and Z are present the following are possible: X═O, Y═O, W═O, Z═O; X═S, Y═O, W═O, Z═O; X═O, Y═S, W═O, Z═O; X═O, Y═O, W═S, Z═O; X═O, Y═O, W═O, Z═S; X═S, Y═S, W═O, Z═O; X═S, Y═O, W═S, Z═O; X═S, Y═O, W═O, Z═S; X═O, Y═O, W═S, Z═S; X═O, Y═S, W═O, Z═S; X═O, Y═S, W═S, Z═O; X═S, Y═S, W═S, Z═O; X═S, Y═S, W═O, Z═S; X═S, Y═O, W═S, Z═S; X═O, Y═S, W═S, Z═S; or X═S, Y═S, W═S, Z═S. In more particular embodiments, the compound has the formula:

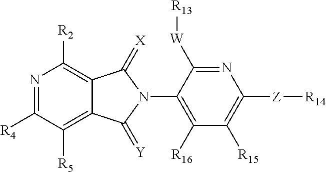

wherein X, Y are independently oxygen or sulfur, and W, Z, R₂, R₄, R₅, and R₁₃-R₁₆ are as before. For example, in more particular embodiments, X or Y is sulfur, and both W and Z are oxygen; both X and Y are sulfur and both W and Z are oxygen; X and Y are both oxygen and W or Z is sulfur; both X and Y are sulfur and W or Z is sulfur; or X or Y are sulfur and both W and Z are sulfur. Alternatively, the following are possible: X═O, Y═O, W═O, Z═O; X═S, Y═O, W═O, Z═O; X═O, Y═S, W═O, Z═O; X═O, Y═O, W═S, Z═O; X═O, Y═O, W═O, Z═S; X═S, Y═S, W═O, Z═O; X═S, Y═O, W═S, Z═O; X═S, Y═O, W═O, Z═S; X═O, Y═O, W═S, Z═S; X═O, Y═S, W═O, Z═S; X═O, Y═S, W═S, Z═O; X═S, Y═S, W═S, Z═O; X═S, Y═S, W═O, Z═S; X═S, Y═O, W═S, Z═S; X═O, Y═S, W═S, Z═S; or X═S, Y═S, W═S, Z═S. In even more particular embodiments, at least one of R₂, R₄, R₅, R₁₅ and R₁₆ is hydroxyl.

Also disclosed is a compound having the formula:

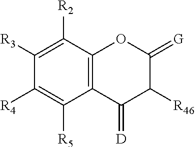

wherein G and D are each independently oxygen or sulfur, R₂-R₅ are as before, and R₄₆ is

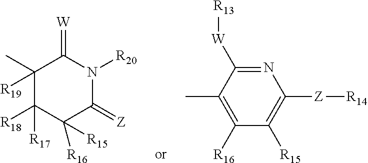

wherein W, Z and R₁₃-R₂₀ are as before. For example, in particular embodiments, G or D is sulfur, and both W and Z are oxygen; both G and D are sulfur and both W and Z are oxygen; G and D are both oxygen and W or Z is sulfur; both G and D are sulfur and W or Z is sulfur; or G or D are sulfur and both W and Z are sulfur. Alternatively, the following are possible: G=O, D=O, W═O, Z═O; G=S, D=O, W═O, Z═O; G=O, D=S, W═O, Z═O; G=O, D=O, W═S, Z═O; G=O, D=O, W═O, Z═S; G=S, D=S, W═O, Z═O; G=S, D=O, W═S, Z═O; G=S, D=O, W═O, Z═S; G=O, D=O, W═S, Z═S; G=O, D=S, W═O, Z═S; G=O, D=S, W═S, Z═O; G=S, D=S, W═S, Z═O; G=S, D=S, W═O, Z═S; G=S, D=O, W═S, Z═S; G=O, D=S, W═S, Z═S; or G=S, D=S, W═S, Z═S.

A method for preventing and/or treating sarcopenia in a subject is also disclosed. The method includes administering to the subject a therapeutically effective amount of one or more of any of the compounds disclosed above, or a compound having the formula:

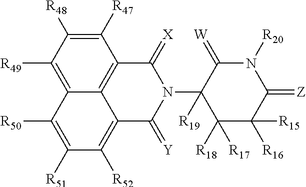

where X and Y are independently oxygen or sulfur; W, Z, R₁₅-R₂₀ are as before; and R₄₇-R₅₂ are each independently hydrogen, hydroxyl, acyl, substituted acyl, acyloxy, substituted acyloxy, alkyl, substituted alkyl, alkenyl, substituted alkenyl, alkynyl, substituted alkynyl, alkoxy, substituted alkoxy, aryl, substituted aryl, amino, substituted amino, halogen or nitro, for example, hydrogen, lower alkyl, acyloxy, halogen, hydroxyl, amino or nitro such as hydrogen, acyloxy or hydroxyl;
or a compound having the formula:

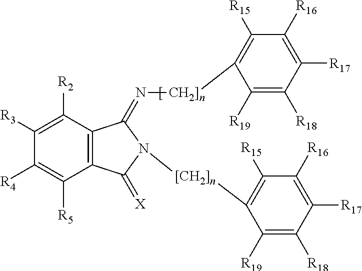

wherein n=1-5; X is oxygen or sulfur, and R₂-R₅ and R₁₅-R₁₉ are as before;
or a compound having the formula:

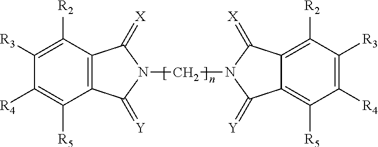

wherein each of X and Y are independently oxygen or sulfur, n=1-5, and R₂-R₅ are as before;
or a compound having the formula:

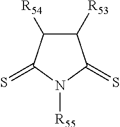

wherein R₅₃ and R₅₄ are independently hydrogen, hydroxyl, acyl, substituted acyl, acyloxy, substituted acyloxy, alkyl, substituted alkyl, alkenyl, substituted alkenyl, alkynyl, substituted alkynyl, alkoxy, substituted alkoxy, aryl, substituted aryl, amino, substituted amino, halogen or nitro, for example, hydrogen, lower alkyl, acyloxy, halogen, hydroxyl, amino or nitro such as hydrogen, acyloxy or hydroxyl; and R₅₅ is hydrogen, alkyl, or substituted alkyl; or a compound having the formula:

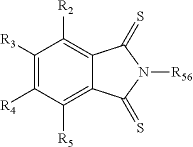

wherein R₂-R₅ are as before and R₅₆ is hydrogen, alkyl or substituted alkyl;
or pharmaceutically acceptable salts or stereoisomers thereof.

Novel thio-substituted analogues having the structures described with respect to the method above also are contemplated. For example, in more particular embodiments, X or Y is sulfur, and both W and Z are oxygen if present; both X and Y are sulfur and both W and Z are oxygen if present; X and Y are both oxygen and W or Z is sulfur if present; both X and Y are sulfur and W or Z is sulfur if present; or X or Y are sulfur and both W and Z are sulfur if present. Alternatively, if W and Z are present, the following are possible: X═O, Y═O, W═O, Z═O; X═S, Y═O, W═O, Z═O; X═O, Y═S, W═O, Z═O; X═O, Y═O, W═S, Z═O; X═O, Y═O, W═O, Z═S; X═S, Y═S, W═O, Z═O; X═S, Y═O, W═S, Z═O; X═S, Y═O, W═O, Z═S; X═O, Y═O, W═S, Z═S; X═O, Y═S, W═O, Z═S; X═O, Y═S, W═S, Z═O; X═S, Y═S, W═S, Z═O; X═S, Y═S, W═O, Z═S; X═S, Y═O, W═S, Z═S; X═O, Y═S, W═S, Z═S; or X═S, Y═S, W═S, Z═S.

Particularly disclosed compounds and compounds that can be used in the disclosed methods include one or more compounds having the following structures:

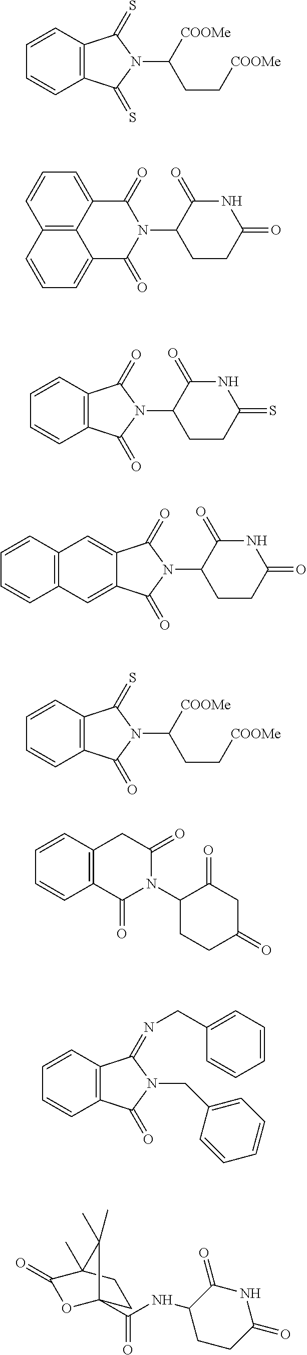

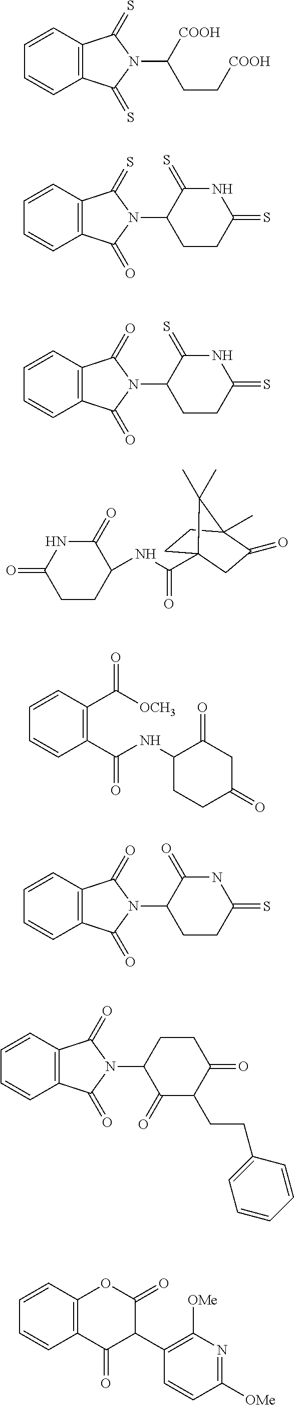

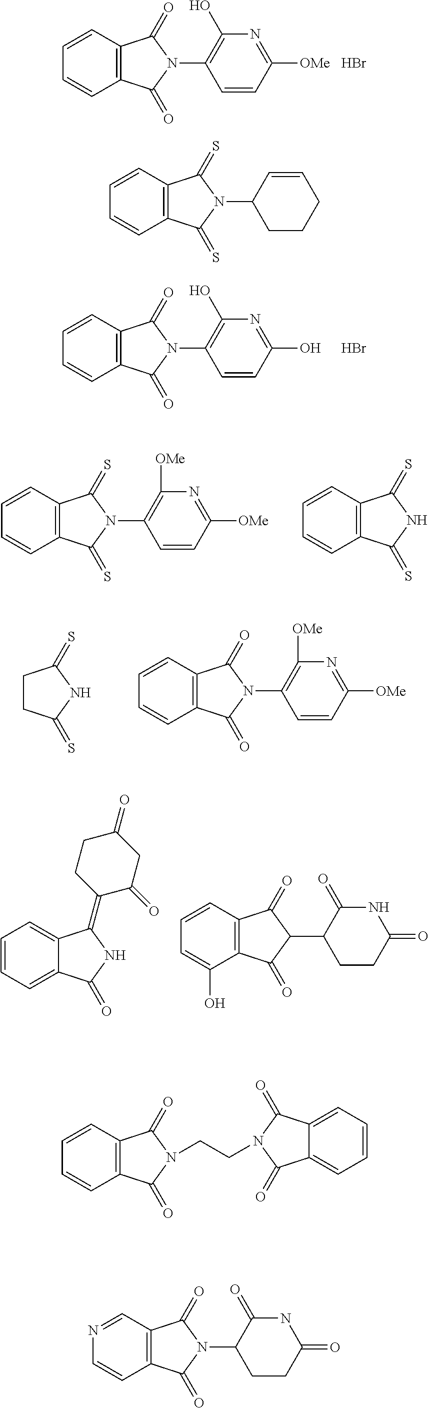

1. A method of maintaining or increasing muscle mass to treat sarcopenia, comprising: administering to a patient in need thereof a therapeutically effective amount of thalidomide or an analogue of thalidomide.

2. The method of claim 1, wherein the patient has a t-score selected from (a) ≦−3, (b) ≦−2.5, (c) ≦−2, (d) ≦−1.5, (e) ≦−1.0 and (f) ≦−0.5

3. The method of claim 1, wherein the age of the patient is selected from at least (a) 40, (b) 50, (c) 55, (d) 60, (e) 65 and (f) 70

4. The method of claim 1, wherein the age range of the patient is selected from (a) 40-50, (b) 50-60 and (c) 60-70

5. The method of claim 1, wherein the patient's t-score is increased after at least one year of treatment.

6. The method of claim 1, wherein the patient's t-score is unchanged after at least one year of treatment.

7. The method of claim 1, wherein the patient has suffered some loss of muscle mass, but does not suffer from a condition that interferes with acts of daily living and/or prevents the subject from living an independent life.

8. The method of claim 1, wherein the patient is considered vulnerable to developing sarcopenia.

9. The method of claim 8, wherein the patient falls into at least one of the following categories (a) uses glucocorticoid steroids, (b) has a chronic infection, (c) has a chronic inflammatory condition and (d) has cancer.

10. A method of maintaining or increasing muscle mass to treat sarcopenia, comprising, administering to a patient in need thereof, a therapeutically effective amount of a pharmaceutical composition, comprising: thalidomide, or an analogue of thalidomide and a pharmaceutically acceptable carrier.

11. The method of claim 1 wherein administration of thalidomide or its analogues produces an effect selected from among stimulation pulsatile growth hormone release; improved bone strength, muscle strength and/or tone; reduced subcutaneous fat in a subject; increased athletic performance; attenuation or reversal of protein catabolic responses following trauma; improved sleep quality; correction of the relative hyposomatotropism of senescence due to high increase in REM sleep and a decrease in REM latency; modification of lipid profile; correction of female androgen deficiency; and correction of male androgen decline.
